(19) United States
(12) Patent Application Publication
(10) Pub. No.: US 2006/0179500 A1
(43) Pub. Date: Aug. 10, 2006

(54) Methods and vectors for improving nucleic acid expression

(75) Inventors: Harry M. Meade, Newton, MA (US); Yann Echelard, Jamaica Plains, MA (US)

Correspondence Address: GTC BIOTHERAPEUTICS, INC., 175 CROSSING BOULEVARD, SUITE 410, FRAMINGHAM, MA, 01702, US

(73) Assignee: GTC-Biotherapeutics, Inc.

(21) Appl. No.: 11/403,633

(22) Filed: Apr. 13, 2006

Related U.S. Application Data
(62) Division of application No. 10/137,618, filed on May 2, 2002.
Application No. 10/137,618 is a continuation-in-part of application No. 09/336,058, filed on Jun. 18, 1999, now abandoned.
(60) Provisional application No. 60/089,918, filed on Jun. 19, 1998.

(51) Int. Cl. A01K 67/027

(52) U.S. Cl. 800/14; 800/21

(57) ABSTRACT

The invention features methods of making transgenic animals, and transgenic animals made by such methods. The method includes introducing into a cell, a nucleic acid construct comprising a nucleic acid sequence encoding a heterologous polypeptide under the control of a mammary epithelial cell promoter, an insulator positioned 5′ from the promoter, an insulator positioned 3′ from the nucleic acid sequence encoding the polypeptide, and a prokaryotic sequence, wherein the sequence between the insulator 5′ from the promoter and the insulator 3′ from the nucleic acid encoding the polypeptide is substantially free of prokaryotic sequence; and allowing a transgenic mammal to develop from the cell, to thereby provide a transgenic mammal.

This application is a continuation-in-part of U.S. Ser. No. 09/336,058 filed on Jun. 18, 1999 which claims priority to U.S. application No. 60/089,918, filed Jun. 18, 1998, the contents of which are incorporated herein by reference.

### SUMMARY

The present invention is based, in part, upon the discovery that insulator sequences can be used to protect a heterologous nucleic acid sequence contained within a vector from the effect of prokaryotic sequence also present within the vector. It has been found that the presence of prokaryotic sequence, e.g., in a vector, used for, e.g., gene therapy or production of transgenic organisms, can negatively impact the expression of heterologous, eukaryotic nucleic acids also contained within the vector. By flanking the heterologous nucleic acid with insulator sequences to form a cassette and then introducing the cassette into a vector, e.g., a vector which includes prokaryotic sequence, expression of the heterologous nucleic acid sequence is improved compared to expression without the insulator sequences. Thus, the present invention allows for use of nucleic acid sequences which include prokaryotic sequence, e.g., in applications were it is burdensome to cut the vector and separate the eukaryotic and prokaryotic sequences. This is true for mass transfections of cell lines such as when a vector is used in cloning methods. This is also the case for gene therapy where it is not possible to grow up a plasmid in a prokaryotic cell such as E. coli and then, in mass, purify the eukaryotic sequence from the prokaryotic sequence. The invention can also reduce the steps required to prepare a transgenic animal, e.g., by microinjection, by eliminating need to cleave the transgene cassette from the plasmid prior to introducing the cassette into an embryo.

Accordingly, in one aspect, the invention features a method of treating a subject having a disorder characterized by a deficiency of a polypeptide. In one embodiment, the method includes administering to the subject a cell comprising a nucleic acid construct. The nucleic acid construct includes a nucleic acid sequence encoding a polypeptide, e.g., a heterologous, eukaryotic polypeptide, under the control of a promoter, an insulator sequence positioned 5′ from the promoter, and an insulator positioned 3′ from the nucleic acid sequence encoding the polypeptide, wherein the nucleic acid construct expresses the encoded polypeptide such that the disorder is treated. The construct further includes a prokaryotic sequence and, e.g., the sequence between the insulator position 5′ from the promoter and the insulator positioned 3′ from the nucleic acid sequence is substantially free of prokaryotic sequence. In a preferred embodiment, the cell is autologous, allogenic or xenogenic cell.

In another embodiment, the method includes administering to the subject a nucleic acid construct. The nucleic acid construct includes a nucleic acid sequence encoding a polypeptide, e.g., a heterologous, eukaryotic polypeptide, under the control of a promoter, an insulator sequence positioned 5′ from the promoter, and an insulator positioned 3′ from the nucleic acid sequence encoding the polypeptide, wherein the nucleic acid construct expresses the encoded polypeptide such that the disorder is treated. The construct further includes a prokaryotic sequence and, e.g., the sequence between the insulator position 5′ from the promoter and the insulator positioned 3′ from the nucleic acid sequence encoding the polypeptide is substantially free of prokaryotic sequence.

In a preferred embodiment, the subject is a human.

In a preferred embodiment, one or more of the insulator sequences is β-globin insulator sequence, e.g., chicken β-globin insulator sequence. Preferably, the insulator 5′ of the promoter and the insulator 3′ of the nucleic acid sequence encoding the polypeptide are β-globin insulator sequences, e.g., chicken β-globin insulator sequences.

In a preferred embodiment, there is 3 kb, 2 kb, 1 kb, 0.5 kb, 0.1 kb or less of prokaryotic sequence between the insulator positioned 5′ from the promoter and the insulator sequence positioned 3′ from the nucleic acid sequence encoding the polypeptide. In another preferred embodiment, there is no prokaryotic sequence between the insulator positioned 5′ from the promoter and the insulator sequence positioned 3′ from the nucleic acid sequence encoding the polypeptide.

In one embodiment, the construct includes: two or more insulator sequences 5′ from the promoter; two or more insulator sequences 3′ from the nucleic acid sequence encoding the polypeptide; two or more insulator sequences 5′ from the promoter and two or more insulator sequences 3′ from the nucleic acid sequence encoding the polypeptide.

In a preferred embodiment, the nucleic acid sequence encoding the polypeptide is a genomic sequence or a cDNA sequence.

In a preferred embodiment, the DNA sequence encodes a therapeutically active polypeptide, e.g., any of: alpha-1 antitrypsin, α-1 proteinase inhibitor, angiogenin, cystic fibrosis transduction regulator (CFTR), erythropoietin, extracellular superoxide dismutase, Factor VIII or other blood factors, fibrinogen, follicle stimulating hormone (FSH), glucocerebrosidase or other proteins associated with a lysosomal storage disorder, granulocyte colony stimulating factor (GCSF), granulocyte macrophage colony stimulating factor (GM-CSF), glutamate decarboxylase, human serum albumin, an immunoglobulin or portion thereof, myelin basic protein, proinsulin, soluble CD4, tissue plasminogen activator, human growth factor, antithrombin III, insulin, and prolactin.

In a preferred embodiment, use of the nucleic acid construct described herein results in higher levels of expression of the polypeptide in the subject as compared to the expression levels when the same nucleic acid construct without the insulator sequences is used in a subject. Preferably, the nucleic acid construct described herein results in at least a 2, 3, 5, 10, 25, 50, 75, 100 fold increase in expression of the polypeptide as compared to the expression levels for such a construct that does not contain the insulator sequences.

In another aspect, the invention features a nucleic acid construct which includes a nucleic acid sequence encoding a heterologous polypeptide (e.g., a heterologous eukaryotic polypeptide) under the control of a promoter, an insulator sequence positioned 5′ from the promoter, and an insulator positioned 3′ from the nucleic acid sequence encoding the polypeptide. The nucleic acid construct further includes a prokaryotic sequence and the sequence between the insulator 5′ from the promoter and the insulator 3′ from the nucleic acid sequence encoding the polypeptide is substantially free of prokaryotic sequence.

In a preferred embodiment, one or more of the insulator sequences is β-globin insulator sequence, e.g., chicken β-globin insulator sequence. Preferably, the insulator 5′ of the promoter and the insulator 3′ of the nucleic acid sequence encoding the polypeptide are β-globin insulator sequences, e.g., chicken β-globin insulator sequences.

In a preferred embodiment, there is 3 kb, 2 kb, 1 kb, 0.5 kb, 0.1 kb or less of prokaryotic sequence between the insulator positioned 5′ from the promoter and the insulator sequence positioned 3′ from the nucleic acid sequence encoding the polypeptide. In another preferred embodiment, there is no prokaryotic sequence between the insulator positioned 5′ from the promoter and the insulator sequence positioned 3′ from the nucleic acid sequence encoding the polypeptide.

In a preferred embodiment, the construct includes: two or more insulator sequences 5′ from the promoter; two or more insulator sequences 3′ from the nucleic acid sequence encoding the polypeptide; two or more insulator sequences 5′ from the promoter and two or more insulator sequences 3′ from the nucleic acid sequence encoding the polypeptide.

In a preferred embodiment, the nucleic acid sequence encoding the polypeptide is a genomic sequence or a cDNA sequence.

In a preferred embodiment, the nucleic acid sequence encodes a therapeutically active polypeptide, e.g., any of: alpha-1 antitrypsin, α-1 proteinase inhibitor, angiogenin, cystic fibrosis transduction regulator (CFTR), erythropoietin, extracellular superoxide dismutase, Factor VIII or other blood factors, fibrinogen, follicle stimulating hormone (FSH), glucocerebrosidase or other proteins associated with a lysosomal storage disorder, granulocyte colony stimulating factor (GCSF), granulocyte macrophage colony stimulating factor (GM-CSF), glutamate decarboxylase, human serum albumin, an immunoglobulin or portion thereof, myelin basic protein, proinsulin, soluble CD4, tissue plasminogen activator, human growth factor, antithrombin III, insulin, and prolactin.

In another aspect, the invention features a method of obtaining expression of a heterologous polypeptide in the milk of a transgenic mammal. The method includes: introducing into a cell, a nucleic acid construct comprising a nucleic acid sequence encoding a heterologous polypeptide (e.g., a heterologous eukaryotic polypeptide) under the control of a mammary epithelial cell promoter, an insulator positioned 5′ from the promoter, an insulator positioned 3′ from the nucleic acid sequence encoding the polypeptide, and a prokaryotic sequence, wherein the sequence between the insulator 5′ from the promoter and the insulator 3′ from the nucleic acid encoding the polypeptide is substantially free of prokaryotic sequence. The method further includes allowing a transgenic mammal to develop from the cell and, milking the transgenic mammal to thereby obtain the heterologous polypeptide.

In a preferred embodiment, the milk comprises about 0.1, 0.5, 1, 5, 10, 25, 50, 100, 500 milligrams per milliliter of the polypeptide. Preferably, the mammal expresses the heterologous polypeptide in its milk at levels at least 2, 3, 5, 10, 50, 100 times greater than mammals prepared using the same construct except without the insulator sequences.

In a preferred embodiment, the construct includes: two or more insulator sequences positioned 5′ from the promoter; two or more insulator sequences positioned 3′ from the nucleic acid sequence encoding the polypeptide; two or more insulator sequences positioned 5′ from the promoter and positioned 3′ from the nucleic acid sequence encoding the polypeptide.

In a preferred embodiment, one or more of the insulator sequences is a β-globin sequence, e.g., chicken β-globin sequence. Preferably, all of the insulator sequences are β-globin sequences, e.g., chicken β-globin sequences.

In a preferred embodiment, the construct has 3 kb, 2 kb, 1 kb, 0.5 kb, 0.1 kb or less of prokaryotic sequence between the insulator positioned 5′ from the promoter and the insulator sequence positioned 3′ from the nucleic acid sequence encoding the polypeptide. In another preferred embodiment, the construct has no prokaryotic sequence between the insulator positioned 5′ from the promoter and the insulator sequence positioned 3′ from the nucleic acid sequence encoding the polypeptide.

In a preferred embodiment, the construct includes any of the following milk specific promoters: a casein promoter, a beta lactoglobulin promoter, a whey acid protein promoter and a lactalbumin promoter.

In a preferred embodiment, the nucleic acid sequence encoding the polypeptide is a genomic sequence or a cDNA sequence.

In a preferred embodiment, the nucleic acid sequence encodes a therapeutically active polypeptide, e.g., any of: α-1 proteinase inhibitor, alkaline phosphates, angiogenin, cystic fibrosis transduction regulator (CFTR) or other membrane proteins, decorin, extracellular superoxide dismutase, fibrinogen, glucocerebrosidase or other proteins associated with lysosomal storage disorders, glutamate decarboxylase, human serum albumin, an immunoglobulin or portion thereof, myelin basic protein, proinsulin, soluble CD4, erythropoietin, tissue plasminogen activator, human growth factor, antithrombin III, insulin, prolactin, a viral protein (e.g., MSP-1), α1-antitrypsin as well as functional fragments of such proteins.

In a preferred embodiment, the method further includes purifying the polypeptide from the milk.

In a preferred embodiment, the mammal is: a cow, a goat, a sheep, a rabbit, a mouse, a pig, or a horse.

In a preferred embodiment, the cell is an oocyte, a one cell embryo, a cell which can be used as a donor cell for nuclear transfer (e.g., an embryonic stem cell, a germ cell, a cell committed to somatic cell lineage, a somatic cell (e.g., a fibroblast)).

In another aspect, the invention features a transgenic non-human mammal whose somatic and germ cells include a nucleic acid sequence comprising a nucleic acid sequence encoding a heterologous polypeptide under the control of a mammary epithelial cell promoter, an insulator sequence positioned 5′ from the promoter, an insulator sequence positioned 3′ from the nucleic acid sequence encoding the polypeptide, and a prokaryotic sequence, wherein the sequence between the insulator 5′ from the promoter and the insulator 3′ from the nucleic acid sequence encoding the polypeptide is substantially free of prokaryotic sequence.

In a preferred embodiment, the mammal express the polypeptide in its milk at about 0.1, 0.5, 1, 5, 10, 25, 50, 100, 500 milligrams per milliliter of the polypeptide. Preferably, the mammal expresses the heterologous polypeptide in its milk at levels at least 2, 3, 5, 10, 50, 100 times greater than mammals that contain the same nucleic acid sequence in their somatic and germ cells except for the insulator sequences.

In a preferred embodiment, the nucleic acid sequence includes: two or more insulator sequences positioned 5′ from the promoter; two or more insulator sequences positioned 3′ from the nucleic acid sequence encoding the polypeptide; two or more insulator sequences positioned 5′ from the promoter and positioned 3′ from the nucleic acid sequence encoding the polypeptide.

In a preferred embodiment, one or more of the insulator sequences are a β-globin sequence, e.g., chicken β-globin sequence. Preferably, all of the insulator sequences are β-globin sequences, e.g., chicken β-globin sequences.

In a preferred embodiment, the nucleic acid sequence has 3 kb, 2 kb, 1 kb, 0.5 kb, 0.1 kb or less of prokaryotic sequence between the insulator positioned 5′ from the promoter and the insulator sequence positioned 3′ from the nucleic acid sequence encoding the polypeptide. In another preferred embodiment, the nucleic acid sequence has no prokaryotic sequence between the insulator positioned 5′ from the promoter and the insulator sequence positioned 3′ from the nucleic acid sequence encoding the polypeptide. Preferably, the prokaryotic sequence is positioned: 5′ to the insulator sequence which is 5′ from the promoter (e.g., the prokaryotic sequence is adjacent to the insulator or less than 1 kb, 0.5 kb, 0.1 kb from the insulator); 3′ to the insulator sequence which is 3′ from the nucleic acid sequence encoding the polypeptide (e.g., the prokaryotic sequence is adjacent to the insulator or less than 1 kb, 0.5 kb, 0.1 kb from the insulator); both 5′ to the insulator which is 5′ from the promoter (e.g., the prokaryotic sequence is adjacent to the insulator or less than 1 kb, 0.5 kb, 0.1 kb from the insulator) and 3′ to the insulator sequence which is 3′ from the nucleic acid sequence encoding the polypeptide (e.g., the prokaryotic sequence is adjacent to the insulator or less than 1 kb, 0.5 kb, 0.1 kb from the insulator).

In a preferred embodiment, the promoter can be any of the following milk specific promoters: a casein promoter, a beta lactoglobulin promoter, a whey acid protein promoter and a lactalbumin promoter.

In a preferred embodiment, the nucleic acid sequence encoding the polypeptide is a genomic sequence or a cDNA sequence.

In a preferred embodiment, the nucleic acid sequence encodes a therapeutically active polypeptide, e.g., any of: α-1 proteinase inhibitor, alkaline phosphates, angiogenin, cystic fibrosis transduction regulator (CFTR) or other membrane proteins, decorin, extracellular superoxide dismutase, fibrinogen, glucocerebrosidase or other proteins associated with lysosomal storage disorders, glutamate decarboxylase, human serum albumin, an immunoglobulin or portion thereof, myelin basic protein, proinsulin, soluble CD4, erythropoietin, tissue plasminogen activator, human growth factor, antithrombin III, insulin, prolactin, a viral protein (e.g., MSP-1), α1-antitrypsin as well as functional fragments of such proteins.

In a preferred embodiment, the mammal is: a cow, a goat, a sheep, a rabbit, a mouse, a pig, or a horse.

The invention also features progeny of such mammals.

In another aspect, the invention features a method of making a transgenic non-human mammal. The method includes: introducing into a cell, a nucleic acid construct comprising a nucleic acid sequence encoding a heterologous polypeptide (e.g., a heterologous eukaryotic polypeptide) under the control of a mammary epithelial cell promoter, an insulator positioned 5′ from the promoter, an insulator positioned 3′ from the nucleic acid sequence encoding the polypeptide, and a prokaryotic sequence, wherein the sequence between the insulator 5′ from the promoter and the insulator 3′ from the nucleic acid encoding the polypeptide is substantially free of prokaryotic sequence. The method further includes allowing a transgenic mammal to develop from the cell, to thereby provide a transgenic mammal.

In a preferred embodiment, the mammal is capable of expressing the polypeptide in its milk at about 0.1, 0.5, 1, 5, 10, 25, 50, 100, 500 milligrams per milliliter of the polypeptide. Preferably, the mammal expresses the heterologous polypeptide in its milk at levels at least 2, 3, 5, 10, 50, 100 times greater than mammals prepared using the same construct except without the insulator sequences.

In a preferred embodiment, the construct includes: two or more insulator sequences positioned 5′ from the promoter; two or more insulator sequences positioned 3′ from the nucleic acid sequence encoding the polypeptide; two or more insulator sequences positioned 5′ from the promoter and positioned 3′ from the nucleic acid sequence encoding the polypeptide.

In a preferred embodiment, one or more of the insulator sequences is a β-globin sequence, e.g., chicken β-globin sequence. Preferably, all of the insulator sequences are β-globin sequences, e.g., chicken β-globin sequences.

In a preferred embodiment, the construct has 3 kb, 2 kb, 1 kb, 0.5 kb, 0.1 kb or less of prokaryotic sequence between the insulator positioned 5′ from the promoter and the insulator sequence positioned 3′ from the nucleic acid sequence encoding the polypeptide. In another preferred embodiment, the construct has no prokaryotic sequence between the insulator positioned 5′ from the promoter and the insulator sequence positioned 3′ from the nucleic acid sequence encoding the polypeptide.

In a preferred embodiment, the construct includes any of the following milk specific promoters: a casein promoter, a beta lactoglobulin promoter, a whey acid protein promoter and a lactalbumin promoter.

In a preferred embodiment, the nucleic acid sequence encoding the polypeptide is a genomic sequence or a cDNA sequence.

In a preferred embodiment, the nucleic acid sequence encodes a therapeutically active polypeptide, e.g., any of: α-1 proteinase inhibitor, alkaline phosphates, angiogenin, cystic fibrosis transduction regulator (CFTR) or other membrane proteins, decorin, extracellular superoxide dismutase, fibrinogen, glucocerebrosidase or other proteins associated with lysosomal storage disorders, glutamate decarboxylase, human serum albumin, an immunoglobulin or portion thereof, myelin basic protein, proinsulin, soluble CD4, erythropoietin, tissue plasminogen activator, human growth factor, antithrombin III, insulin, prolactin, a viral protein (e.g., MSP-1), α1-antitrypsin as well as functional fragments of such proteins.

In a preferred embodiment, the mammal is: a cow, a goat, a sheep, a rabbit, a mouse, a pig, or a horse.

In a preferred embodiment, the cell is an oocyte, a one cell embryo, a cell which can be used as a donor cell for nuclear transfer (e.g., an embryonic stem cell, a germ cell, a cell committed to somatic cell lineage, a somatic cell (e.g., a fibroblast)).

In another aspect, the invention features a method of obtaining expression of a heterologous polypeptide in the milk of a transgenic mammal which includes providing a mammal or the progeny of a mammal produced by the methods described herein, and obtaining milk from the mammal.

In another aspect, the invention features a transgenic non-human animal whose somatic and germ cells include a nucleic acid sequence comprising a nucleic acid sequence encoding a heterologous polypeptide under the control of a promoter, an insulator sequence positioned 5′ from the promoter, an insulator sequence positioned 3′ from the nucleic acid sequence encoding the polypeptide, and a prokaryotic sequence, wherein the sequence between the insulator 5′ from the promoter and the insulator 3′ from the nucleic acid sequence encoding the polypeptide is substantially free of prokaryotic sequence.

In a preferred embodiment, the animal expresses the heterologous polypeptide at levels at least 2, 3, 5, 10, 50, 100 times greater than animals that contain the same nucleic acid sequence in their somatic and germ cells except for the insulator sequences.

In a preferred embodiment, the nucleic acid sequence includes: two or more insulator sequences positioned 5′ from the promoter; two or more insulator sequences positioned 3′ from the nucleic acid sequence encoding the polypeptide; two or more insulator sequences positioned 5′ from the promoter and positioned 3′ from the nucleic acid sequence encoding the polypeptide.

In a preferred embodiment, one or more of the insulator sequences are a β-globin sequence, e.g., chicken β-globin sequence. Preferably, all of the insulator sequences are β-globin sequences, e.g., chicken β-globin sequences.

In a preferred embodiment, the nucleic acid sequence has 3 kb, 2 kb, 1 kb, 0.5 kb, 0.1 kb or less of prokaryotic sequence between the insulator positioned 5′ from the promoter and the insulator sequence positioned 3′ from the nucleic acid sequence encoding the polypeptide. In another preferred embodiment, the nucleic acid sequence has no prokaryotic sequence between the insulator positioned 5′ from the promoter and the insulator sequence positioned 3′ from the nucleic acid sequence encoding the polypeptide. Preferably, the prokaryotic sequence is positioned: 5′ to the insulator sequence which is 5′ from the promoter (e.g., the prokaryotic sequence is adjacent to the insulator or less than 1 kb, 0.5 kb, 0.1 kb from the insulator); 3′ to the insulator sequence which is 3′ from the nucleic acid sequence encoding the polypeptide (e.g., the prokaryotic sequence is adjacent to the insulator or less than 1 kb, 0.5 kb, 0.1 kb from the insulator); both 5′ to the insulator which is 5′ from the promoter (e.g., the prokaryotic sequence is adjacent to the insulator or less than 1 kb, 0.5 kb, 0.1 kb from the insulator) and 3′ to the insulator sequence which is 3′ from the nucleic acid sequence encoding the polypeptide (e.g., the prokaryotic sequence is adjacent to the insulator or less than 1 kb, 0.5 kb, 0.1 kb from the insulator).

In a preferred embodiment, the promoter is a tissue specific promoter and, e.g., the polypeptide is expressed in the tissue at levels at least 2, 3, 5, 10, 50, 100 times greater than it is in that tissue of animals that contain the same nucleic acid sequence in their somatic and germ cells except for the insulator sequences. In a preferred embodiment, the promoter can be, e.g., a mammary epithelial cell promoter, or a promoter which preferentially expresses a protein in egg, blood, urine, etc. For example, the promoter can be any of the following mammary epithelial cell promoters: a casein promoter, a beta lactoglobulin promoter, a whey acid protein promoter and a lactalbumin promoter.

In a preferred embodiment, the nucleic acid sequence encoding the polypeptide is a genomic sequence or a cDNA sequence.

In a preferred embodiment, the nucleic acid sequence encodes a therapeutically active polypeptide, e.g., any of: α-1 proteinase inhibitor, alkaline phosphates, angiogenin, cystic fibrosis transduction regulator (CFTR) or other membrane proteins, decorin, extracellular superoxide dismutase, fibrinogen, glucocerebrosidase or other proteins associated with lysosomal storage disorders, glutamate decarboxylase, human serum albumin, an immunoglobulin or portion thereof, myelin basic protein, proinsulin, soluble CD4, erythropoietin, tissue plasminogen activator, human growth factor, antithrombin III, insulin, prolactin, a viral protein (e.g., MSP-1 ), α1-antitrypsin as well as functional fragments of such proteins.

In a preferred embodiment, the animal is a mammal, e.g., a cow, a goat, a sheep, a rabbit, a mouse, a pig, or a horse. In other embodiments, the animal is, e.g., a bird.

The invention also features progeny of such mammals.

In another aspect, the invention features a method of making a transgenic non-human animal. The method includes: introducing into a cell, a nucleic acid construct comprising a nucleic acid sequence encoding a heterologous polypeptide (e.g., a heterologous eukaryotic polypeptide) under the control of a promoter, an insulator positioned 5′ from the promoter, an insulator positioned 3′ from the nucleic acid sequence encoding the polypeptide, and a prokaryotic sequence, wherein the sequence between the insulator 5′ from the promoter and the insulator 3′ from the nucleic acid encoding the polypeptide is substantially free of prokaryotic sequence. The method further includes allowing a transgenic animal to develop from the cell, to thereby provide a transgenic animal.

In a preferred embodiment, the animal expresses the heterologous polypeptide at levels at least 2, 3, 5, 10, 50, 100 times greater than animals that contain the same nucleic acid sequence in their somatic and germ cells except for the insulator sequences.

In a preferred embodiment, the construct includes: two or more insulator sequences positioned 5′ from the promoter; two or more insulator sequences positioned 3′ from the nucleic acid sequence encoding the polypeptide; two or more insulator sequences positioned 5′ from the promoter and positioned 3′ from the nucleic acid sequence encoding the polypeptide.

In a preferred embodiment, one or more of the insulator sequences is a β-globin sequence, e.g., chicken β-globin sequence. Preferably, all of the insulator sequences are β-globin sequences, e.g., chicken β-globin sequences.

In a preferred embodiment, the construct has 3 kb, 2 kb, 1 kb, 0.5 kb, 0.1 kb or less of prokaryotic sequence between the insulator positioned 5′ from the promoter and the insulator sequence positioned 3′ from the nucleic acid sequence encoding the polypeptide. In another preferred embodiment, the construct has no prokaryotic sequence between the insulator positioned 5′ from the promoter and the insulator sequence positioned 3′ from the nucleic acid sequence encoding the polypeptide.

In a preferred embodiment, the promoter is a tissue specific promoter and, e.g., the polypeptide is expressed in the tissue at levels at least 2, 3, 5, 10, 50, 100 times greater than it is in that tissue of animals that contain the same nucleic acid sequence in their somatic and germ cells except for the insulator sequences. In a preferred embodiment, the promoter can be, e.g., a mammary epithelial cell promoter, or a promoter which preferentially expresses a protein in egg, blood, urine, etc. For example, the promoter can be any of the following mammary epithelial cell promoters: a casein promoter, a beta lactoglobulin promoter, a whey acid protein promoter and a lactalbumin promoter.

In a preferred embodiment, the nucleic acid sequence encoding the polypeptide is a genomic sequence or a cDNA sequence.

In a preferred embodiment, the nucleic acid sequence encodes a therapeutically active polypeptide, e.g., any of: α-1 proteinase inhibitor, alkaline phosphates, angiogenin, cystic fibrosis transduction regulator (CFTR) or other membrane proteins, decorin, extracellular superoxide dismutase, fibrinogen, glucocerebrosidase or other proteins associated with lysosomal storage disorders, glutamate decarboxylase, human serum albumin, an immunoglobulin or portion thereof, myelin basic protein, proinsulin, soluble CD4, erythropoietin, tissue plasminogen activator, human growth factor, antithrombin III, insulin, prolactin, a viral protein (e.g., MSP-1), α1-antitrypsin as well as functional fragments of such proteins.

In a preferred embodiment, the animal is a mammal, e.g., a cow, a goat, a sheep, a rabbit, a mouse, a pig, or a horse. In another preferred embodiment, the animal is a bird.

In a preferred embodiment, the cell is an oocyte, a one cell embryo, a cell which can be used as a donor cell for nuclear transfer (e.g., an embryonic stem cell, a germ cell, a cell committed to somatic cell lineage, a somatic cell (e.g., a fibroblast)).

Preferred transgenic animals include: mammals; birds; reptiles; and amphibians. Mammals as used herein refer to non-human mammals including cow, goat, sheep, rabbit, mouse, pigs and horses. Preferred mammals are mammals which produce a large volume of milk and have long lactating periods, e.g., cows, goats and sheep.

The terms “protein”, “polypeptide” and “peptide” are used interchangeably herein.

The term “substantially no prokaryotic sequence” between described points in a nucleic acid sequence means that there is less than 4 kb of prokaryotic sequence within the defined area of the sequence. Preferably, there is less than 3 kb, 2 kb, 1 kb, 0.5 kb, 0.1 kb, 0.05 kb prokaryotic sequence or no prokaryotic sequence present within the defined area of the sequence.

A “heterologous polypeptide” or “exogenous polypeptide” is a polypeptide that normally is not produced by the cell or animal, or is not normally produced in, e.g., the mammary gland or oviduct of an animal (e.g., an antibody normally present only in serum). Alternatively, a heterologous polypeptide or exogenous polypeptide may be produced by the cell or animal under normal conditions, but the level of expression is augmented or enhanced to increase production.

The terms “prokaryotic” and “eukaryotic” are known in the art as referring to microorganisms which have a distinct nucleus (eukaryotic) or do not have a distinct nucleus (prokaryotic). The terms “prokaryotic sequence” and “eukaryotic sequence” as used herein refer to sequences which naturally occur in either a prokaryotic or eukaryotic organism.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows fragments of plasmids BC918, BC917 and BC995. Two fragments were derived from BC918, namely 918 and 918N. Fragment 918 includes a β-casein promoter, a sequence encoding LEA and a 3′ UTR from β-casein. Fragment 918 N includes SuperCos sequence, a β-casein promoter, a sequence encoding LEA and a 3′ UTR from P-casein. Two fragments were derived from BC917, namely 917 and 917 N. Fragment 917 includes a β-globin insulator, a β-casein promoter, a sequence encoding LEA and a 3′ UTR from β-casein. Fragment 917 N includes SuperCos. sequence, a β-globin insulator, a β-casein promoter, a sequence encoding LEA and a 3′ UTR from β-casein a β-globin insulator.

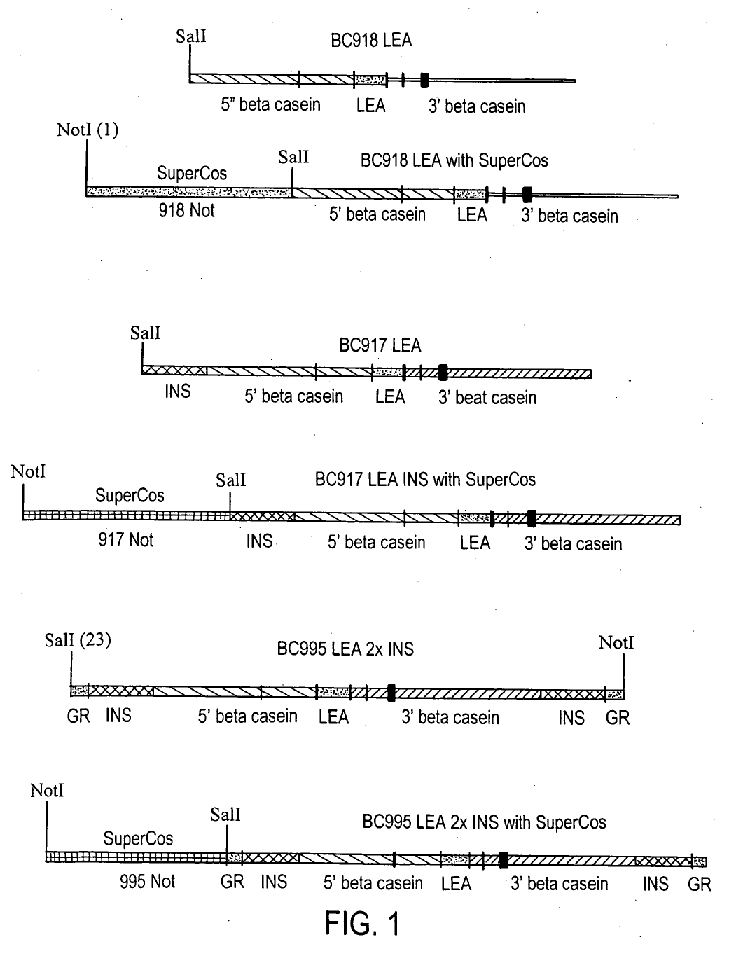

### DETAILED DESCRIPTION

Transfected Host Cells

Genetically engineered host cells can be derived from a cell line into which a nucleic acid of interest, e.g., a nucleic acid which encodes a protein, has been introduced. For example, a nucleic acid molecule within a vector can be introduced into a host cell.

A host cell can be a eukaryotic or prokaryotic cell. For example, a polypeptide of interest can be expressed in bacterial cells such as E. coli, insect cells, yeast cells or mammalian cells, e.g., Chinese hamster ovary cells (CHO) or COS cells. In addition, the heterologous polypeptide can be expressed in a transgenic animal or a specific tissue of a transgenic animal, e.g., mammary epithelial cells. In one aspect, a nucleic acid construct which includes at least one insulator sequence (preferably, at least two insulator sequences, one positioned 5′ from the promoter and one positioned 3′ from the nucleic acid encoding the polypeptide) and a nucleotide sequence encoding a polypeptide of interest under the control of a promoter is included in a vector, e.g., a prokaryotic plasmid, and introduced into a host cell. Preferably, at least one of the promoter or nucleic acid encoding the polypeptide is a eukaryotic sequence. The host cell can be, e.g., E. coli, or a cells used to produce a transgenic animal (e.g., an donor cell for nuclear transfer). By flanking the eukaryotic sequence or sequences with insulator sequences, this region is protected from the prokaryotic sequence of the plasmid.

A construct can be introduced into a cell via conventional transformation or transfection techniques. As used herein, the terms “transfection” and “transformation” include a variety of techniques for introducing a transgenic sequence into a host cell, including calcium phosphate or calcium chloride co-precipitation, DEAE-dextrane-mediated transfection, lipofection, or electroporation. In addition, biological vectors, e.g., viral vectors can be used as described below. Suitable methods for transforming or transfecting host cells can be found in Sambrook et al., Molecular Cloning: A Laboratory Manuel, 2^{nd} ed., Cold Spring Harbor Laboratory, (Cold Spring Harbor Laboratory Press, Cold Spring Harbor, N.Y., 1989), and other suitable laboratory manuals.

Two useful approaches are electroporation and lipofection. Brief examples of each are described below.

The nucleic acid construct can be stably introduced into a cell by electroporation using the following protocol: the cells are resuspended in PBS at about 4×10⁶ cells/ml. Fifty micorgrams of linearized DNA is added to the 0.5 ml cell suspension, and the suspension is placed in a 0.4 cm electrode gap cuvette (Biorad). Electroporation is performed using a Biorad Gene Pulser electroporator with a 330 volt pulse at 25 mA, 1000 microFarad and infinite resistance. If the nucleic acid construct contains a Neomyocin resistance gene for selection, neomyocin resistant clones are selected following incubation with 350 microgram/ml of G418 (GibcoBRL) for 15 days.

The nucleic acid construct can be stably introduced into a cell by lipofection using a protocol such as the following: about 2×10⁵ cells are plated into a 3.5 cmiameter well and transfected with 2 micrograms of linearized DNA using LipfectAMINE™ (GibcoBRL). Forty-eight hours after transfection, the cells are split 1:1000 and 1:5000 and, if the DNA construct contains a neomyosin resistance gene for selection, G418 is added to a final concentration of 0.35 mg/ml. Neomyocin resistant clones are isolated and expanded for cyropreservation as well as nuclear transfer.

In addition, a nucleic acid or nucleic acid construct can be introduced into a host cell by microinjection. For example, the transgene can be introduced into a mammal by microinjection of the construct into the pronuclei of the fertilized mammalian egg(s) to cause one or more copies of the construct to be retained in the cells of the developing mammal(s). Following introduction of the transgene construct into the fertilized egg, the egg may be incubated in vitro for varying amounts of time, or reimplanted into the surrogate host, or both. One common method is to incubate the embryos in vitro for about 1-7 days, depending on the species, and then reimplant them into the surrogate host.

The DNA can also be introduced into a host cell by cloning procedures. For example, the genome of a genetically engineered cell (e.g., somatic cell) can be fused with a donor oocyte to form a nuclear transfer unit. Such methods are described, for example, in PCT Publication WO 97/07668 and PCT Publication WO 97/07669, the contents of which are incorporated herein by reference.

The progeny of the transgenically manipulated embryos may be tested for the presence of the construct by Southern blot analysis of the segment of tissue. If one or more copies of the exogenous cloned construct remains stably integrated into the genome of such transgenic embryos, it is possible to establish permanent transgenic mammal lines carrying the transgenically added construct.

Promoters

A promoter can be selected based upon the expression system contemplated. This can include promoters used for general expression of a polypeptide and promoters which preferentially express a polypeptide in a particular tissue.

For example, general promoters can be used for expression in several different tissues. Examples of general promoters include β-actin, ROSA-21, PGK, FOS, c-myc, Jun-A, Jun-B, CMV promoter and elongation factor-1α promoter.

Tissue-Specific Expression of Proteins

It is often desirable to express a protein, e.g., a heterologous protein, in a specific tissue or fluid, e.g., the milk, blood, urine, eggs, of a transgenic animal. The heterologous protein can be recovered from the tissue or fluid in which it is expressed. For example, it is often desirable to express the heterologous protein in milk. Methods for producing a heterologous protein under the control of a milk specific promoter are described below. In addition, other tissue-specific promoters, as well as, other regulatory elements, e.g., signal sequences and sequence which enhance secretion of non-secreted proteins, are described below.

Milk Specific Promoters

Useful transcriptional promoters are those promoters that are preferentially activated in mammary epithelial cells, including promoters that control the genes encoding milk proteins such as caseins, beta lactoglobulin (Clark et al., (1989) Bio/Technology 7: 487-492), whey acid protein (Gordon et al. (1987) Bio/Technology 5: 1183-1187),-and lactalbumin (Soulier et al., (1992) FEBS Letts. 297: 13). Casein promoters may be derived from the alpha, beta, gamma or kappa casein genes of any mammalian species. A preferred promoter is derived from the goat beta casein gene (DiTullio, (1992) Bio/Technology 10:74-77). Milk-specific protein promoter or the promoters that are specifically activated in mammary tissue can be derived from cDNA or genomic sequences. Preferably, they are genomic in origin.

DNA sequence information is available for the mammary gland specific genes listed above, in at least one, and often in several organisms. See, e.g., Richards et al., J. Biol. Chem. 256, 526-532 (1981) (α-lactalbumin rat); Campbell et al., Nucleic Acids Res. 12, 8685-8697 (1984) (rat WAP); Jones et al., J. Biol. Chem. 260, 7042-7050 (1985) (rat β-casein); Yu-Lee & Rosen, J. Biol. Chem. 258, 10794-10804 (1983) (rat γ-casein); Hall, Biochem. J. 242, 735-742 (1987) (α-lactalbumin human); Stewart, Nucleic Acids Res. 12, 389 (1984) (bovine αs1 and κ casein cDNAs); Gorodetsky et al., Gene 66, 87-96 (1988) (bovine β casein); Alexander et al., Eur. J. Biochem. 178, 395-401 (1988) (bovine κ casein); Brignon et al., FEBS Lett. 188, 48-55 (1977) (bovine αS2 casein); Jamieson et al., Gene 61, 85-90 (1987), Ivanov et al., Biol. Chem. Hoppe-Seyler 369, 425-429 (1988), Alexander et al., Nucleic Acids Res. 17, 6739 (1989) (bovine β lactoglobulin); Vilotte et al., Biochimie 69, 609-620 (1987) (bovine α-lactalbumin). The structure and function of the various milk protein genes are reviewed by Mercier & Vilotte, J. Dairy Sci. 76, 3079-3098 (1993) (incorporated by reference in its entirety for all purposes). If additional flanking sequence are useful in optimizing expression of the heterologous protein, such sequences can be cloned using the existing sequences as probes. Mammary-gland specific regulatory sequences from different organisms can be obtained by screening libraries from such organisms using known cognate nucleotide sequences, or antibodies to cognate proteins as probes.

Other Tissue-Specific Promoters

Other tissue-specific promoters which provide expression in a particular tissue can be used. Tissue specific promoters are promoters which are expressed more strongly in a particular tissue than in others. Tissue specific promoters are often expressed essentially exclusively in the specific tissue.

Tissue-specific promoters which can be used include: a neural-specific promoter, e.g., nestin, Wnt-1, Pax-1, Engrailed-1, Engrailed-2, Sonic hedgehog; a liver-specific promoter, e.g., albumin, alpha-1 antitrypsin; a muscle-specific promoter, e.g., myogenin, actin, MyoD, myosin; an oocyte specific promoter, e.g., ZP1, ZP2, ZP3; a testes-specific promoter, e.g., protamin, fertilin, synaptonemal complex protein-1; a blood-specific promoter, e.g., globulin, GATA-1, porphobilinogen dearninase; a lung-specific promoter, e.g., surfactant protein C; a skin- or wool-specific promoter, e.g., keratin, elastin; endothelium-specific promoters, e.g., Tie-1, Tie-2; and a bone-specific promoter, e.g., BMP.

Signal Sequences

Any known signal sequence can be used. Useful signal sequences include milk-specific signal sequences as well as other signal sequences which result in the secretion of eukaryotic or prokaryotic proteins. Preferably, for milk production, the signal sequence is selected from milk-specific signal sequences, i.e., it is from a gene which encodes a product secreted into milk. Most preferably, the milk-specific signal sequence is related to the milk-specific promoter used in the construct, which are described below. The size of the signal sequence is not critical. All that is required is that the sequence be of a sufficient size to effect secretion of the desired recombinant protein, e.g., in the mammary tissue. For example, signal sequences from genes coding for caseins, e.g., alpha, beta, gamma or kappa caseins, beta lactoglobulin, whey acid protein, and lactalbumin can be used. A preferred signal sequence is the goat β-casein signal sequence.

Signal sequences from other secreted proteins, e.g., proteins secreted by kidney cells, pancreatic cells or liver cells, can also be used. Preferably, the signal sequence results in the secretion of proteins into, for example, urine, blood or eggs.

Insulator Sequences

The DNA constructs of the invention further comprise at least one insulator sequence. The terms “insulator”, “insulator sequence” and “insulator element” are used interchangeably herein. An insulator element is a control element which insulates the transcription of genes placed within its range of action but which does not perturb gene expression, either negatively or positively. Preferably, an insulator sequence is inserted on either side of the DNA sequence to be transcribed. For example, the insulator can be positioned about 200 bp to about 1 kb, 5′ from the promoter, and at least about 1 kb to 5 kb from the promoter, at the 3′ end of the gene of interest. The distance of the insulator sequence from the promoter and the 3′ end of the gene of interest can be determined by those skilled in the art, depending on the relative sizes of the gene of interest, the promoter and the enhancer used in the construct. In addition, more than one insulator sequence can be positioned 5′ from the promoter or at the 3′ end of the transgene. For example, two or more insulator sequences can be positioned 5′ from the promoter. The insulator or insulators at the 3′ end of the transgene can be positioned at the 3′ end of the gene of interest, or at the 3′ end of a 3′ regulatory sequence, e.g., a 3′ untranslated region (UTR) or a 3′ flanking sequence.

A preferred insulator is a DNA segment which encompasses the 5′ end of the chicken β-globin locus and corresponds to the chicken 5′ constitutive hypersensitive site as described in PCT Publication 94/23046, the contents of which is incorporated herein by reference.

DNA Constructs

A cassette which encodes a heterologous protein can be assembled as a construct which includes an insulator sequence (e.g., a β globin insulator, e.g., a chicken β globin insulator), a promoter, e.g., for a specific tissue (e.g., for mammary epithelial cells, e.g., a casein promoter, e.g., a goat beta casein promoter), a signal sequence (e.g., a milk-specific signal sequence, e.g., a casein signal sequence, e.g., a β-casein signal sequence), and a nucleic acid sequence encoding the heterologous protein.

The cassette can also include a 3′ UTR downstream of the nucleic acid sequence coding for the heterologous protein. Such regions can stabilize the RNA transcript of the expression system and thus increases the yield of desired protein from the expression system. Among the 3′ untranslated regions useful in the constructs of the invention are sequences that provide a poly A signal. Such sequences may be derived, e.g., from the SV40 small t antigen, the casein 3′ untranslated region or other 3′ untranslated sequences well known in the art. In one aspect, the 3′ untranslated region is derived from a milk specific protein. The length of the 3′ untranslated region is not critical but the stabilizing effect of its poly A transcript appears important in stabilizing the RNA of the expression sequence. Other 3′ flanking regions can also be included in the cassette.

Optionally, the cassette can include a 5′ UTR between the promoter and the nucleic acid sequence encoding the signal sequence. Such untranslated regions can be from the same control region from which promoter is taken or can be from a different gene, e.g., they may be derived from other synthetic, semi-synthetic or natural sources. Again their specific length is not critical, however, they appear to be useful in improving the level of expression. The cassette can also include 5′ flanking sequences.

Expression Vectors

Preferably, the cassette is part of a vector. The portions of the cassette can be introduced together or separately into the vector.

Expression vectors, containing a nucleic acid encoding a polypeptide of interest (or a portion thereof) can be introduced into a host cell. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

A recombinant expression vector can comprise a nucleic acid encoding a polypeptide of interest in a form suitable for expression of the nucleic acid in a host cell. This means that the recombinant expression vectors can include one or more of the regulatory elements discussed above. For example, the vector can include a promoter, enhancer, and/or signal sequence operatively linked to the nucleotide sequence encoding the polypeptide of interest. As discussed above, regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors can be introduced into host cells to thereby produce proteins or peptides encoded by a nucleic acid molecule.

The recombinant expression vector can be designed for expression of a polypeptide of interest in prokaryotic (e.g., E. coli) or eukaryotic cells (e.g., insect cells (using baculovirus expression vectors), yeast cells or mammalian cells).

Expression of proteins in prokaryotes is most often carried out in E. coli with vectors containing constitutive or inducible promoters directing the expression of the polypeptide of interest.

In other embodiments, the expression vector can be a yeast expression vector, a baculovirus expression vector or a mammalian expression vector. Examples of vectors for expression in yeast S. cerivisae include pYepSec1 (Baldari et al. (1987) EMBO J. 6:229-234), pMFa (Kujan and Herskowitz, (1982) Cell 30:933-943), pJRY88 (Schultz et al. (1987) Gene 54:113-123), pYES2 (Invitrogen Corporation, San Diego, Calif.), and pPicZ (Invitrogen Corp, San Diego, Calif.). Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39). Examples of mammalian expression vectors include pCDM8 (Seed (1987) Nature 329:840) and pMT2PC (Kaufinan et al. (1987) EMBO J. 6:187-195).

Heterologous Proteins

The invention can be used to express various different polypeptides.

In one aspect, transgenic sequences encoding heterolbgous proteins can be introduced into the germline of a non-human mammal or can be transfected into a cell. A heterologous protein can be a protein from a different gene that the promoter or can be a protein from another species.

The protein can be a complex or multimeric protein, e.g., a homo- or heteromultimer, e.g., proteins which naturally occur as homo- or heteromultimers, e.g., homo- or hetero-dimers, trimers or tetramers. The protein can be a protein which is processed by removal, e.g., cleavage, of N-terminus, C-terminus or internal fragments. Even complex proteins can be expressed in active form. Protein encoding sequences which can be introduced into the genome of mammal, e.g., goats, include glycoproteins, neuropeptides, immunoglobulins, enzymes, peptides and hormones. The protein may be a naturally occurring protein or a recombinant protein, e.g., a fragment, fusion protein, e.g., an immunoglogulin fusion protein, or mutien. It may be human or non-human in origin. The heterologous protein may be a potential therapeutic or pharmaceutical agent such as, but not limited to: alpha-1 proteinase inhibitor, alpha-1 antitrypsine, alkaline phosphatase, angiogenin, antithrombin III, any of the blood clotting factors including Factor VIII, Factor IX, and Factor X chitinase, decorin, erythropoietin, extracellular superoxide dismutase, fibrinogen, glucocerebrosidase, glutamate decarboxylase, human growth factor, human serum albumin, immunoglobulin, insulin, myelin basic protein, proinsulin, prolactin, soluble CD4 or a component or complex thereof, lactoferrin, lactoglobulin, lysozyme, lactalbumin, tissue plasminogen activator or variants thereof.

Immunoglobulins are particularly preferred heterologous proteins. Examples of immunoglobulins include IgA, IgG, IgE, IgM, chimeric antibodies, humanized antibodies, recombinant antibodies, single chain antibodies and antibody-protein fusions.

Nucleotide sequence information is available for several of the genes encoding the heterologous proteins listed above, in at least one, and often in several organisms. See e.g., Long et al. (1984) Biochem. 23(21):4828-4837 (aplha-1 antitrypsin); Mitchell et al. (1986) Prot. Natl. Acad. Sci USA 83:7182-7186 (alkaline phosphatase); Schneider et al. (1988) EMBO J. 7(13):4151-4156 (angiogenin); Bock et al. (1988) Biochem. 27(16):6171-6178 (antithrombin III); Olds et al. (1991) Br. J. Haematol. 78(3):408-413 (antithrombin III); Lin et al. (1985) Proc. Natl. Acad. Sci. USA 82(22):7580-7584 (erythropoeitin); U.S. Pat. No. 5,614,184 (erythropoietin); Horowitz et al. (1989) Genomics 4(1):87-96 (glucocerebrosidase); Kelly et al. (1992) Ann. Hum. Genet. 56(3):255-265 (glutamte decarboxylase); U.S. Pat. No. 5,707,828 (human serum albumin); U.S. Pat. No. 5,652,352 (human serum albumin); Lawn et al. (1981) Nucleic Acid Res. 9(22):6103-6114 (human serum albumin); Kamholz et al. (1986) Prot. Natl. Acad. Sci. USA 83(13):4962-4966 (myelin basic protein); Hiraoka et al. (1991) Mol. Cell Endocrinol. 75(1):71-80 (prolactin); U.S. Pat. No. 5,571,896 (lactoferrin); Pennica et al. (1983) Nature 301(5897):214-221 (tissue plasminogen activator); Sarafanov et al. (1995) Mol. Biol. 29:161-165, the contents of which are incorporated herein by reference.

Purification of Proteins from Milk

The transgenic protein can be produced in milk at relatively high concentrations and in large volumes, providing continuous high level output of normally processed peptide that is easily harvested from a renewable resource. There are several different methods known in the art for isolation of proteins form milk.

Milk proteins usually are isolated by a combination of processes. Raw milk first is fractionated to remove fats, for example, by skimming, centrifugation, sedimentation (H. E. Swaisgood, Developments in Dairy Chemistry, I: Chemistry of Milk Protein, Applied Science Publishers, NY, 1982), acid precipitation (U.S. Pat. No. 4,644,056) or enzymatic coagulation with rennin or chymotrypsin (Swaisgood, ibid.). Next, the major milk proteins may be fractionated into either a clear solution or a bulk precipitate from which the specific protein of interest may be readily purified.

French Patent No. 2487642 describes the isolation of milk proteins from skim milk or whey by membrane ultrafiltration in combination with exclusion chromatography or ion exchange chromatography. Whey is first produced by removing the casein by coagulation with rennet or lactic acid. U.S. Pat. No. 4,485,040 describes the isolation of an alpha-lactoglobulin-enriched product in the retentate from whey by two sequential ultrafiltration steps. U.S. Pat. No. 4,644,056 provides a method for purifying immunoglobulin from milk or colostrum by acid precipitation at pH 4.0-5.5, and sequential cross-flow filtration first on a membrane with 0.1-1.2 micrometer pore size to clarify the product pool and then on a membrane with a separation limit of 5-80 kd to concentrate it.

Similarly, U.S. Pat. No. 4,897,465 teaches the concentration and enrichment of a protein such as immunoglobulin from blood serum, egg yolks or whey by sequential ultrafiltration on metallic oxide membranes with a pH shift. Filtration is carried out first at a pH below the isoelectric point (pI) of the selected protein to remove bulk contaminants from the protein retentate, and next at a pH above the pI of the selected protein to retain impurities and pass the selected protein to the permeate. A different filtration concentration method is taught by European Patent No. EP 467 482 B 1 in which defatted skim milk is reduced to pH 3-4, below the pI of the milk proteins, to solubilize both casein and whey proteins. Three successive rounds of ultrafiltration or diafiltration then concentrate the proteins to form a retentate containing 15-20% solids of which 90% is protein. Alternatively, British Patent Application No. 2179947 discloses the isolation of lactoferrin from whey by ultrafiltration to concentrate the sample, followed by weak cation exchange chromatography at approximately a neutral pH. No measure of purity is reported. In PCT Publication No. WO 95/22258, a protein such as -lactoferrin is recovered from milk that has been adjusted to high ionic strength by the addition of concentrated salt, followed by cation exchange chromatography.

In all of these methods, milk or a fraction thereof is first treated to remove fats, lipids, and other particulate matter that would foul filtration membranes or chromatography media. The initial fractions thus produced may consist of casein, whey, or total milk protein, from which the protein of interest is then isolated.

PCT Patent Publication No. WO 94/19935 discloses a method of isolating a biologically active protein from whole milk by stabilizing the solubility of total milk proteins with a positively charged agent such as arginine, imidazole or Bis-Tris. This treatment forms a clarified solution from which the protein may be isolated, e.g., by filtration through membranes that otherwise would become clogged by precipitated proteins.

U.S. Ser. No. 08/648,235 discloses a method for isolating a soluble milk component, such as a peptide, in its biologically active form from whole milk or a milk fraction by tangential flow filtration. Unlike previous isolation methods, this eliminates the need for a first fractionation of whole milk to remove fat and casein micelles, thereby simplifying the process and avoiding losses of recovery and bioactivity. This method may be used in combination with additional purification steps to further remove contaminants and purify the product, e.g., protein, of interest.

Gene Therapy

The constructs of the invention can also be used as a part of a gene therapy protocol to deliver nucleic acids encoding a polypeptide. The invention features expression vectors for in vivo transfection and expression of a polypeptide in particular cell types so as to reconstitute the function of, or alternatively, antagonize the function of a polypeptide in a cell in which that polypeptide is misexpressed. Expression constructs of polypeptides, may be administered in any biologically effective carrier, e.g. any formulation or composition capable of effectively delivering the gene to cells in vivo. Approaches include insertion of the subject gene in viral vectors including recombinant retroviruses, adenovirus, adeno-associated virus, and herpes simplex virus-1, or recombinant bacterial or eukaryotic plasmids. Viral vectors transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct or CaPO₄ precipitation carried out in vivo.

A preferred approach for in vivo introduction of nucleic acid into a cell is by use of a viral vector containing nucleic acid encoding a polypeptide. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid.

Retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, A. D. (1990) Blood 76:271). A replication defective retrovirus can be packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include ψCrip, ψCre, ψ2 and ψAm. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Another viral gene delivery system useful in the present invention utilizes adenovirus-derived vectors. The genome of an adenovirus can be manipulated such that it encodes and expresses a gene product of interest but is inactivated in terms of its ability to replicate in a normal lytic viral life cycle. See, for example, Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are known to those. skilled in the art. Recombinant adenoviruses can be advantageous in certain circumstances in that they are not capable of infecting nondividing cells and can be used to infect a wide variety of cell types, including epithelial cells (Rosenfeld et al. (1992) cited supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267).

Yet another viral vector system useful for delivery of the subject gene is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al. (1992) Curr. Topics in Micro. and Immunol. 158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

In addition to viral transfer methods, such as those illustrated above, non-viral methods can also be employed to cause expression of a polypeptide in the tissue of an animal. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the subject gene by the targeted cell. Exemplary gene delivery systems of this type include liposomal derived systems, poly-lysine conjugates, and artificial viral envelopes. Other embodiments include plasmid injection systems such as are described in Meuli et al. (2001) J Invest Dermatol. 116(1):131-135; Cohen et al. (2000) Gene Ther 7(22):1896-905; or Tam et al. (2000) Gene Ther 7(21):1867-74.

In a representative embodiment, a gene encoding a polypeptide can be entrapped in liposomes bearing positive charges on their surface (e.g., lipofectins) and (optionally) which are tagged with antibodies against cell surface antigens of the target tissue (Mizuno et al. (1992) No Shinkei Geka 20:547-551; PCT publication WO91/06309; Japanese patent application 1047381; and European patent publication EP-A-43075).

In clinical settings, the gene delivery systems for the therapeutic gene can be introduced into a patient by any of a number of methods, each of which is familiar in the art. For instance, a pharmaceutical preparation of the gene delivery system can be introduced systemically, e.g. by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g. Chen et al. (1994) PNAS 91: 3054-3057).

The pharmaceutical preparation of the gene therapy construct can consist essentially of the gene delivery system in an acceptable diluent, or can comprise a slow release matrix in which the gene delivery vehicle is imbedded. Alternatively, where the complete gene delivery system can be produced in tact from recombinant cells, e.g. retroviral vectors, the pharmaceutical preparation can comprise one or more cells which produce the gene delivery system.

Cell Therapy

The polypeptide can also be increased in a subject by introducing into a cell, e.g., a fibroblast, a promoter sequence; an enhancer sequence, e.g., 5′ untranslated region (UTR); a nucleotide sequence encoding a polypeptide or functional fragment or analog thereof; a 3′ UTR; a polyadenylation site; at least two insulator sequences. The cell can then be introduced into the subject.

Primary and secondary cells to be genetically engineered can be obtained form a variety of tissues and include cell types which can be maintained propagated in culture. For example, primary and secondary cells include fibroblasts, keratinocytes, epithelial cells (e.g., mammary epithelial cells, intestinal epithelial cells), endothelial cells, glial cells, neural cells, formed elements of the blood (e.g., lymphocytes, bone marrow cells), muscle cells (myoblasts) and precursors of these somatic cell types. Primary cells are preferably obtained from the individual to whom the genetically engineered primary or secondary cells are administered. However, primary cells may be obtained for a donor (other than the recipient) of the same species or another species (e.g., mouse, rat, rabbit, cat, dog, pig, cow, bird, sheep, goat, horse).

The term “primary cell” includes cells present in a suspension of cells isolated from a vertebrate tissue source (prior to their being plated i.e., attached to a tissue culture substrate such as a dish or flask), cells present in an explant derived from tissue, both of the previous types of cells plated for the first time, and cell suspensions derived from these plated cells. The term “secondary cell” or “cell strain” refers to cells at all subsequent steps in culturing. That is, the first time a plated primary cell is removed from the culture substrate and replated (passaged), it is referred to herein as a secondary cell, as are all cells in subsequent passages. Secondary cells are cell strains which consist of secondary cells which have been passaged one or more times. A cell strain consists of secondary cells that: 1) have been passaged one or more times; 2) exhibit a finite number of mean population doublings in culture; 3) exhibit the properties of contact-inhibited, anchorage dependent growth (anchorage-dependence does not apply to cells that are propagated in suspension culture); and 4) are not immortalized. A “clonal cell strain” is defined as a cell strain that is derived from a single founder cell. A “heterogenous cell strain” is defined as a cell strain that is derived from two or more founder cells.

Primary or secondary cells of vertebrate, particularly mammalian, origin can be transfected with an exogenous nucleic acid sequence which includes a nucleic acid sequence encoding a heterologous nucleic acid sequence, and produce the encoded product stably and reproducibly in vitro and in vivo, over extended periods of time.

The transfected primary or secondary cells may also include DNA encoding a selectable marker which confers a selectable phenotype upon them, facilitating their identification and isolation. Methods for producing transfected primary and secondary cells which stably express exogenous synthetic DNA, clonal cell strains and heterogeneous cell strains of such transfected cells, methods of producing the clonal heterogeneous cell strains, and methods of treating or preventing an abnormal or undesirable condition through the use of populations of transfected primary or secondary cells are part of the present invention.

Transfection of Primary or Secondary Cells of Clonal or Heterogeneous Cell Strains

Vertebrate tissue can be obtained by standard methods such a punch biopsy or other surgical methods of obtaining a tissue source of the primary cell type of interest. For example, punch biopsy is used to obtain skin as a source of fibroblasts or keratinocytes. A mixture of primary cells is obtained from the tissue, using known methods, such as enzymatic digestion or explanting. If enzymatic digestion is used, enzymes such as collagenase, hyaluronidase, dispase, pronase, trypsin, elastase and chymotrypsin can be used.

The resulting primary cell mixture can be transfected directly or it can be cultured first, removed from the culture plate and resuspended before transfection is carried out. Primary cells or secondary cells are combined with exogenous nucleic acid sequence to, e.g., stably integrate into their genomes, and treated in order to accomplish transfection. The exogenous nucleic acid sequence can optionally include DNA encoding a selectable marker. The exogenous nucleic acid sequence and selectable marker-encoding DNA can either be on separate constructs or on a single construct. An appropriate quantity of DNA is used to ensure that at least one stably transfected cell containing and appropriately expressing exogenous DNA is produced. In general, approximately 0.1 to 500 μg of DNA is used.

As used herein, the term “transfection” includes a variety of techniques for introducing an exogenous nucleic acid into a cell including calcium phosphate or calcium chloride precipitation, microinjection, DEAE-dextrin-mediated transfection, lipofection or electrophoration.

Electroporation is carried out at approximate voltage and capacitance (and corresponding time constant) to result in entry of the DNA construct(s) into the primary or secondary cells. Electroporation can be carried out over a wide range of voltages (e.g., 50 to 2000 volts) and corresponding capacitance. Total DNA of approximately 0.1 to 500 μg is generally used.

Methods such as calcium phosphate precipitation, modified calcium phosphate precipitation an polybrene precipitation, liposome fusion and receptor-mediated gene delivery can also be used to transect cells. Primary or secondary cells can also be transfected using microinjection. A stably, transfected cell can then be isolated and cultured and sub cultivated, under culturing conditions and for sufficient time to propagate stably transfected secondary cells an produce a clonal cell strain of transfected secondary cells. Alternatively, more than one transfected cell is cultured and sub cultured, resulting in production of a heterogeneous cell strain.

Transfected primary or secondary cells undergo sufficient number doubling to produce either a clonal cell strain or a heterogeneous cell strain of sufficient size to provide the therapeutic protein to an individual in effective amounts. In general, for example, 0.1 cm² of skin is biopsies and assumed to contain 1,000,000 cells; one cell is used to produce a clonal cell strain and undergoes approximately 27 doublings to produce 100 million transfected secondary cells. If a heterogeneous cell strain is to be produced from an original transfected population of approximately 1000,000 cells, only 10 doublings are needed to produce 100 million transfected cells.

The number of required cells in a transfected clonal heterogeneous cell strain is variable and depends on a variety of factors, including but not limited to, the use of the transfected cells, the functional level of the exogenous DNA in the transfected cells, the site of implantation of the transfected cells (for example, the number of cells that can be used is limited by the anatomical site of implantation), and the age, surface area, and clinical condition of the patient. The put these factors in perspective, to deliver therapeutic levels of human growth hormone in an otherwise healthy 10 kg patient with isolated growth hormone deficiency, approximately one to five hundred million transfected fibroblast would be necessary (the volume of these cells is about that of the very tip of the patient's thumb).

Implantation of Clonal Cell Strain or Heterogeneous Cell Strain of Transfected Secondary Cells

The transfected cells, e.g., cells produced as described herein, can be introduced into an individual to whom the product is to be delivered. The clonal cell strain or heterogeneous cell strain is then introduced into an individual. Various routed of administration and various sites (e.g., renal sub capsular, subcutaneous, central nervous system (including intrathecal), intravascular, intrahepatic, intrasplanchnic, intraperitoneal (including intraomental), intramuscularly implantation) can be used. One implanted in individual, the transfected cells produce the product encoded by the heterologous DNA or are affected by the heterologous DNA itself. For example, an individual who suffers hemophilia may be a candidate for cells expressing Factor VIII.

The individual can have a small skin biopsy performed; this is a simple procedure which can be performed on an outpatient basis. The piece of skin is taken, for example, from under the arn and can require about one minute to remove. The sample is processed, resulting in isolation of the patient's cell (e.g., fibroblasts) and genetically engineered to produce the polypeptide of interest. Based on the age, weight, and clinical condition of the patient, the required number of cells are grown in large-scale culture. The entire process should require 4-6 weeks and, at the end of that time, the appropriate number of genetically engineered cells are introduced into the individual, once again as an outpatient (e.g., by injecting them back under the patient's skin, e.g., on the scalp or face). The patient is now capable of producing the polypeptide, e.g., Factor VIII which can ameliorate symptoms of hemphilia.

For some, this will be a one-time treatment and, for others, multiple cell therapy treatments will be required.

As this example suggests, the cells used will generally be patient-specific genetically engineered cells. It is possible, however, to obtain cells from another individual of the same species or from a different species. Use of such cells might require administration of an immunosuppressant, alteration of histocompatibility antigens, or use of a barrier device to prevent rejection of the implanted cells.

Transfected primary or secondary cells can be administered alone or in conjunction with a barrier or agent for inhibiting immune response against the cell in a recipient subject. For example, an immunosuppressive agent can be administered to a subject to inhibit or interfere with normal response in the subject. Preferably, the immunosuppressive agent is an immunosuppressive drug which inhibits T cell/or B cell activity in a subject. Examples of such immunosuppressive drugs commercially available (e.g., cyclosporin A is commercially avail for Sandoz Corp. East Hanover, N.J.).

An immunosuppressive agent e.g., drug, can be administered to a subject at a dosage sufficient to achieve the desired therapeutic effect (e.g., inhibition of rejection of the cells). Dosage ranges for immunosuppressive drugs are known in the art. See, e.g., Freed et al. (1992) N. Engl. J. Med. 327:1549; Spencer et al. (1992) N. Engl. J. Med. 327:154′ Widner et al. (1992) n. Engl. J. Med. 327:1556). Dosage values may vary according to factors such as the disease state, age, sex, and weight of the individual.

Another agent with can be used to inhibit T cell activity in a subject is an antibody, or fragment of derivative thereof. Antibodies capable of depleting or sequestering T cells in vivo are known in the art. Polyclonal antisera can be used, for example, anti-lymphocyte serum. Alternatively, one or more monoclonal antibodies can be used. Preferred T cell depleting antibodies include monoclonal antibodies which bind to CD2, CD3, CD4, CD8, CD40, CD40, ligand on the cell surface. Such antibodies are known in the art and are commercially available, for example, form American Type Culture Collection. A preferred antibody for binding CD3 on human T cells is OKT3 (ATCC CRL 8001).

An antibody which depletes, sequesters or inhibits T cells within a recipient subject can be administered in a dose for an appropriate time to inhibit rejection of cells upon transplantation. Antibodies are preferably administered intravenously in a pharmaceutically acceptable carrier of diluent (e.g., saline solution).

Another way of interfering with or inhibiting an immune response to the cells in a recipient subject is to use an inununobarrier. An “immunobarrier” as used herein, refers to a device which serves as a barrier between the administered cell and cells involved in immune response in a subject. For example, the cells can be administered in an implantable device. An implantable device can include the cells contained within a semi-permeable barrier, i.e., one which lets nutrients and the product diffuse in and out of the barrier but which prevents entry or larger immune system components, e.g., antibodies or complement. An implant able device typically includes a matrix, e.g., a hydrogel, biocompatible mesh, or core in which cells are disposed. Optionally, a semi permeable coating can enclose the gel. If disposed within the gel core, the administered cells should be sequestered from the cells of the immune system and should be cloaked from the cells and cytotoxic antibodies of the host. Preferably, a permselective coating such as PLL or PLO is used. The coating often has a porosity which prevents components of the recipient's immune system from entering and destroying the cells within the implantable device.

Many methods for encapsulating cells are known in the art. For example, encapsulation using a water soluble gum to obtain a semi-permeable water insoluble gel to encapsulate cells for production and other methods of encapsulation are disclosed in U.S. Pat. No.: 4,352,883. Other implantable devices which can be used are disclosed in U.S. Pat. No.: 5,084,350, U.S. Pat. No. 5,427.935, WO 95/19743 published Jul. 27, 1995, U.S. Pat. No.: 5,545,423, U.S. Pat. No. 4,409,331, U.S. Pat. No. 4,663,286, and European Patent No. 301,777.

An advantage of the use of transfected or secondary cells is that by controlling the number of cells introduced into an individual, one can control the amount of the protein delivered to the body. In addition, in some cases, it is possible to remove the transfected cells of there is no longer a need for the product. A further advantage of treatment by use of transfected primary or secondary cells of the present invention is that production of the therapeutic product can be regulated, such as through the an administration of zinc, steroids or an agent which affects transcription of a protein, product or nucleic acid product or affects the stability of a nucleic acid product.

This invention is further illustrated by the following examples which in no way should be construed as being further limiting.

### EXAMPLE

### Example 1

### Expression of LEA in the Milk of Transgenic Mice

This experiment demonstrates that the insulators protect the expression of a transgene within a vector from influences of the prokaryotic sequences within the vector. This was done by generating transgenic mice that carried transgenes with and without insulators flanking the expression cassette. As shown in FIG. 4, various constructs were made with or without insulator sequences, and with or without supercos (prokaryotic) sequence being present. Briefly, the cassettes are as follows: 918 (inject fragment) is a fragment of BC918 which includes (in order) a beta casein promoter, a nucleic acid sequence encoding LEA and a 3′ untranslated region from beta-casein; 918 N (Scos linked) is the same as 918 except that it includes a SuperCos sequence 5′ to the beta casein promoter; 917 (injected fragment) is a fragment of BC917 which includes (in order) an insulator sequence, a beta casein promoter, a nucleic acid sequence encoding LEA and a 3′ untranslated region from beta-casein; 917 N (Scos linked) is the same as 917 except that it includes a Supercos sequence 5′ to the first insulator sequence; 995 (injected fragment) is a fragment of BC918 which includes (in order) an insulator sequence, a beta casein promoter, a nucleic acid sequence encoding LEA, a 3′ untranslated region from beta-casein, and an insulator sequence; 995 N (Scos linked) is the same as 995 2×INS except that it includes a SuperCos sequence 5′ to the first insulator sequence.

The goal was to show that 995 N (Scos linked) can expressed at greater levels than 918 N (Scos linked) which includes no insulator sequences.

The results are provided in Table I below:

TABLE I

| | | Average Expression | |
|---|---|---|---|
| | Number of | Levels of the Mice | |
| Construct | Mice Expressing | (mg/ml) | |
| 918 (injected fragment) | 1 of 2 | <.3 | mg/ml |
| 918 N (Scos linked) | 0 of 8 | 0.012 | mg/ml |
| 917 INS (injected frag.) | 7 of 7 | <1.13 | mg/ml |
| 917 INS N (Scos linked) | 1 of 8 | 0.075 | mg/ml |
| 995 2× INS (inj frag) | 4 of 5 | 0.2 | mg/ml |
| 995 N(Scos linked) | 3 of 6 | 0.15 | mg/ml |

It was found that 995 N, which has SuperCos prokaryotic sequence linked to it, is expressed. The average expression levels of 995N were less than those found in the milk of mice injected with the 995 fragment alone.

A number of embodiments of the invention have been- described. Nevertheless, it will be understood that various modifications may be made without departing from the spirit and scope of the invention. Accordingly, other embodiments are within the scope of the following claims.

1-26. (canceled)

27. A method of making a transgenic non-human mammal, comprising:
introducing into a cell, a nucleic acid construct comprising a nucleic acid sequence encoding a heterologous polypeptide under the control of a mammary epithelial cell promoter, an insulator positioned 5′ from the promoter, an insulator positioned 3′ from the nucleic acid sequence encoding the polypeptide, and a prokaryotic sequence, wherein the sequence between the insulator 5′ from the promoter and the insulator 3′ from the nucleic acid encoding the polypeptide is substantially free of prokaryotic sequence;
allowing a transgenic mammal to develop from the cell, to thereby provide a transgenic mammal;
wherein substantially free of prokaryotic sequence means that the nucleic acid sequence has 0.5 kb or less of prokaryotic sequence between the insulator positioned 5′ from the promoter and the insulator sequence positioned 3′ from the nucleic acid sequence encoding the polypeptide; and,
wherein the nucleic acid sequence encoding the heterologous polypeptide under the control of a mammary epithelial cell promoter is genetically engineered into the genome of a somatic cell which is later fused with a enucleated oocyte to form a nuclear transfer unit.

28. The method of claim 27, wherein the mammal is capable of expressing the polypeptide in its milk at about 0.1 milligrams per milliliter of the polypeptide.

29. The method of claim 27, wherein the mammal expresses the heterologous polypeptide in its milk at levels at least 2 times greater than mammals prepared using the same construct except without the insulator sequences.

30. The method of claim 27, wherein the mammal expresses the heterologous polypeptide in its milk at levels at least 5 times greater than mammals prepared using the same construct except without the insulator sequences.

31. The transgenic non-human mammal of claim 27 whose somatic and germ cells comprise a nucleic acid sequence comprising a nucleic acid sequence encoding a heterologous polypeptide under the control of a mammary epithelial cell promoter.

32. The method of claim 27, wherein the construct has 0.5 kb or less of prokaryotic sequence between the insulator positioned 5′ from the promoter and the insulator sequence as positioned 3′ from the nucleic acid sequence encoding the polypeptide.
